(19) United States
(12) Plant Patent Application Publication
(10) Pub. No.: US 2017/0027093 P1
(43) Pub. Date: Jan. 26, 2017

(54) Blueberry plant named 'RIDLEY 4408'

(50) ;

(71) Applicant: Mountain Blue Orchards Pty Ltd., New South Wales (AU)

(72) Inventor: Ridley Bell, New South Wales (AU)

(21) Appl. No.: 14/999,950

(22) Filed: Jul. 18, 2016

Related U.S. Application Data
(60) Provisional application No. 62/282,015, filed on Jul. 22, 2015.

(51) Int. Cl. A01H 5/00

(52) U.S. Cl. PLT/157

(57) ABSTRACT

The new blueberry plant variety ‘RIDLEY 4408’ is provided. The variety is produced from a cross of ‘S01-15-01’ (unpatented) and ‘C00-09’ (U.S. Plant Pat. No. 22,778), which can be distinguished by its outstanding features.

### CROSS REFERENCE TO RELATED APPLICATIONS

This application claims the benefit of U.S. Provisional Patent Application No. 62/282,015 filed on Jul. 22, 2015, which is incorporated herein by reference in its entirety as though fully set forth.

### STATEMENT REGARDING FEDERALLY-SPONSORED RESEARCH AND DEVELOPMENT

None.

### LATIN NAME OF THE FAMILY, GENUS, AND SPECIES

Family—Ericaceae

Genus—Vaccinium

Species—hybrid

### VARIETY DENOMINATION

The new blueberry plant claimed is of the variety denominated ‘RIDLEY 4408’.

### BACKGROUND OF THE INVENTION

The present invention relates to a new and distinct perennial variety of Vaccinium hybrid (Southern Highbush Blueberry), which has been given the variety denomination of ‘Ridley 4408. Its market class is that of a fruiting plant. ‘Ridley 4408’ is intended for use as fresh fruit for shipping, customer pick and processing markets and as a home garden plant.

The new Vaccinium hybrid cultivar is a selection resulting from seedlings produced in a breeding programme of Vaccinium at Lindendale, NSW, Australia in August 2009 from the controlled pollination of seed parent ‘S01-15-01’ (unpatented) with pollen parent ‘C00-09’ (U.S. Plant Pat. No. 22,778). The new cultivar was discovered and selected as a single plant, breeder code M11-44-08, within a population of 100 resulting Vaccinium hybrid plants from this controlled pollination in September 2011 in a commercial field plantation environment at Lindendale, New South Wales, Australia. It was monitored over the next three years with five inspections per year to confirm consistency of traits. Selection criteria were, in combination, suitability to machine harvesting of fresh fruit characterized by firm, round to slightly oblate shaped fruit that drop easily from the bush when shaken leaving the unripe fruit on the bush, medium plant growth vigor, low chilling requirement, early to medium time of flowering and fruit ripening, medium fruit size, medium to high yield, desirable tasting berries and small picking scar.

The selection was evaluated for a number of years at commercial farms at Lindendale, New South Wales, Australia.

Asexual reproduction of the new cultivar by cutting propagation since 2011 at Lindendale, New South Wales, Australia has demonstrated that the new cultivar reproduces true to type with all of the characteristics, as herein described, firmly fixed and retained through successive generations of such asexual propagation.

### SUMMARY OF THE INVENTION

The new Vaccinium hybrid cultivar is a selection, breeder code M11-44-08, resulting from seedlings produced in a breeding programme of Vaccinium at Lindendale, NSW, Australia in August 2009 from the controlled pollination of seed parent ‘S01-15-01’ (unpatented) with pollen parent ‘C00-09’ (U.S. Plant Pat. No. 22,778).

Plants of the new cultivar differ from plants of the seed parent ‘S01-15-01’ primarily in medium plant growth vigor, firm, crisp fruit with a pleasant and strong sweet acid balanced flavour whereas the seed parent has weak plant growth vigor with spindly branches, less firm fruit with an acidic unpleasant flavour. Plants of the new cultivar differ from plants of the pollen parent ‘C00-09’ primarily in medium plant growth vigor, early to medium time of vegetative bud burst, early to medium time of first flowering, early to medium time of first fruit ripening, medium fruit size and round to slightly oblate fruit shape whereas the pollen parent has strong plant growth vigor, late time of vegetative bud burst, late time of first flowering, late time of first fruit ripening, very large fruit size and oblate fruit shape.

The new Vaccinium hybrid cultivar is a selection, breeder code M11-44-08, resulting from seedlings produced in a breeding programme of Vaccinium at Lindendale, NSW, Australia in August 2009 from the controlled pollination of seed parent ‘S01-15-01’ (unpatented) with pollen parent ‘C00-09’ (U.S. Plant Pat. No. 22,778).

Asexual reproduction of the new cultivar by cutting propagation since 2011 at Lindendale, New South Wales, Australia has demonstrated that the new cultivar reproduces true to type with all of the characteristics, as herein described, firmly fixed and retained through successive generations of such asexual propagation.

The new Vaccinium hybrid cultivar is a selection resulting from seedlings produced in a breeding programme of Vaccinium at Lindendale, NSW, Australia in 2009 from the controlled pollination of seed parent ‘S01-15-01’ (unpatented) with pollen parent ‘ C00-09’ (U.S. Plant Pat. No. 22,778). The new cultivar was discovered and selected as a single plant, breeder code M11-44-08, within a population of 100 resulting Vaccinium hybrid plants from this controlled pollination in 2009 in a commercial field plantation environment at Lindendale, New South Wales, Australia. Selection criteria included a combination of medium plant growth vigor, low chilling requirement, early to medium time of flowering and fruit ripening, medium fruit size, round to slightly oblate fruit shape, firm fruit suited to handling, potential for machine harvesting with fruit easily detached by shaking, medium to high yield, desirable tasting berries and small picking scar.

The selection was subsequently evaluated for a number of years at the commercial farms at Lindendale, New South Wales, Australia.

The following characteristics of the new cultivar have been repeatedly observed and can be used to distinguish ‘RIDLEY 4408’ as a new and distinct cultivar of Vaccinium hybrid plant:
1. Upright to semi upright and bushy plant growth habit
2. Early to medium timing of beginning of flowering
3. Early to medium timing of berry ripening
4. Medium plant growth vigor
5. Berry is firm and crisp
6. Picking scar is dry
7. Leaf width is broad
8. Berry size is medium
9. Berry sweetness and acidity are high making desirable flavor
10. Bloom on berry is strong
11. Dark blue berry color

Plants of the new cultivar differ from plants of the seed parent ‘S01-15-01’ primarily in medium plant growth vigor, firm, crisp fruit with a pleasant and strong sweet acid balanced flavour whereas the seed parent has weak plant growth vigor with spindly branches, less firm fruit with an acidic unpleasant flavour. Plants of the new cultivar differ from plants of the pollen parent ‘C00-09’ primarily in medium plant growth vigor, early to medium time of vegetative bud burst, early to medium time of first flowering, early to medium time of first fruit ripening, medium fruit size and round to slightly oblate fruit shape whereas the pollen parent has strong plant growth vigor, late time of vegetative bud burst, late time of first flowering, late time of first fruit ripening, very large fruit size and oblate fruit shape.

Asexual reproduction of the new cultivar by cutting propagation since 2011 at Lindendale, New South Wales, Australia has demonstrated that the new cultivar reproduces true to type with all of the characteristics, as herein described, firmly fixed and retained through successive generations of such asexual propagation.

### BRIEF DESCRIPTION OF THE DRAWINGS

The accompanying photographic illustration shows typical specimens in full color of the foliage and fruit of the new variety ‘Ridley 4408.’ The colors are as nearly true as is reasonably possible in a color representation of this type.

FIG. 1 is a photograph of the new variety ‘Ridley 4408’, demonstrating the plant's growth habit and fruit set.

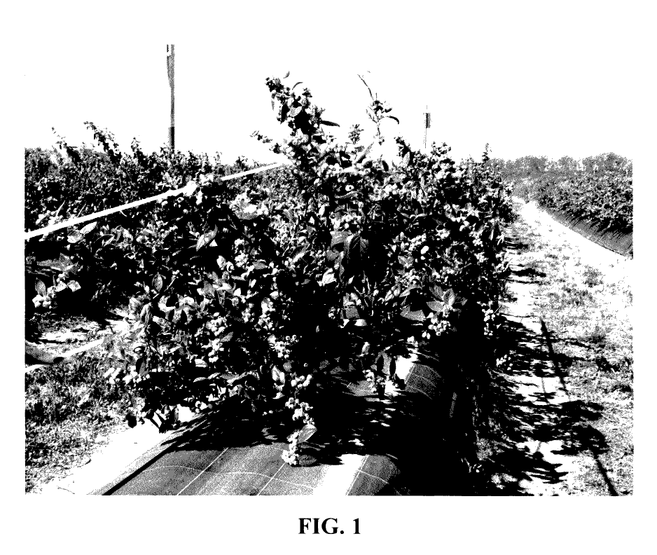

FIG. 2 is a photograph of the flowering plant of the new variety ‘Ridley 4408’.

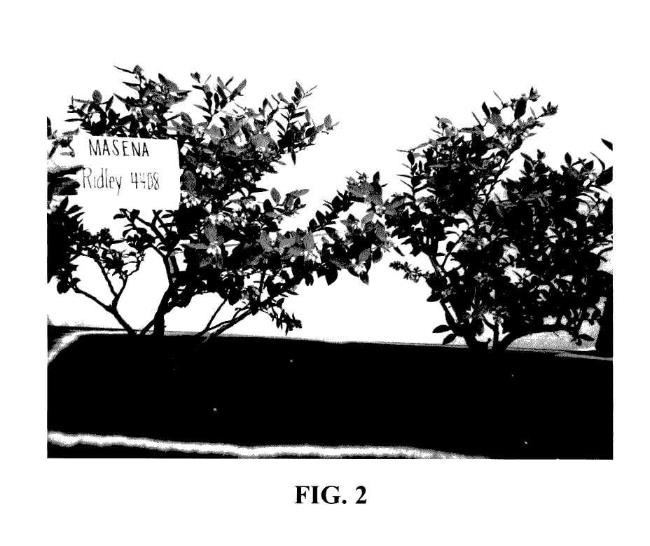

FIG. 3 is a photograph of the leaves of the new variety ‘Ridley 4408’.

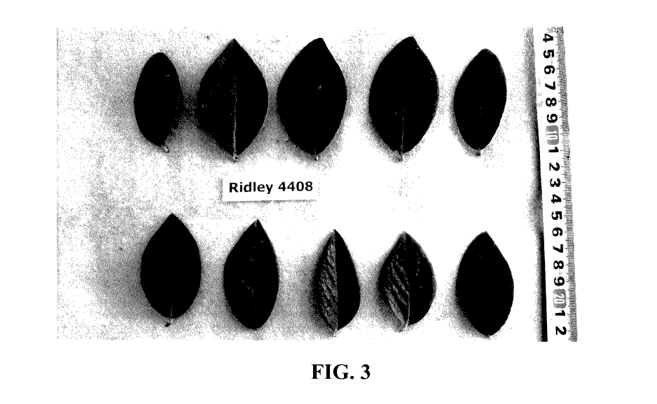

FIG. 4 is a photograph of the flower, leaf and fruit of the new variety ‘Ridley 4408’.

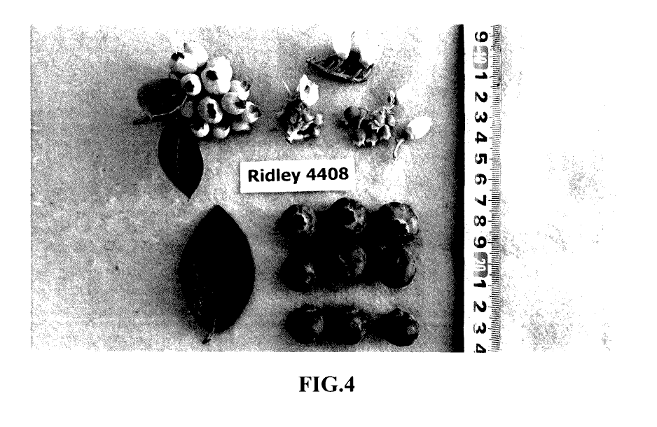

FIG. 5 is a photograph of the inflorescence and flower of the new variety ‘Ridley 4408’.

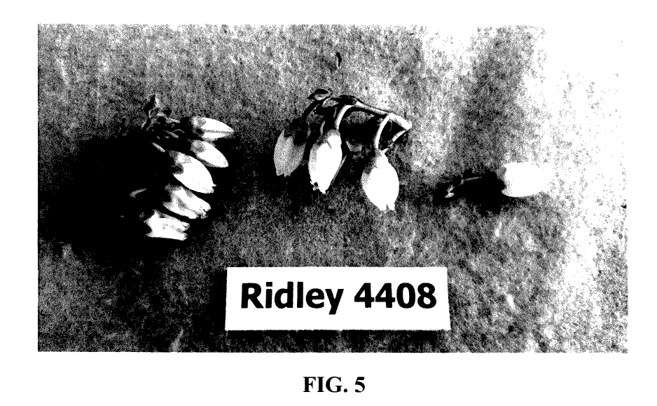

FIG. 6 is a photograph of the fruit detail of the new variety ‘Ridley 4408’.

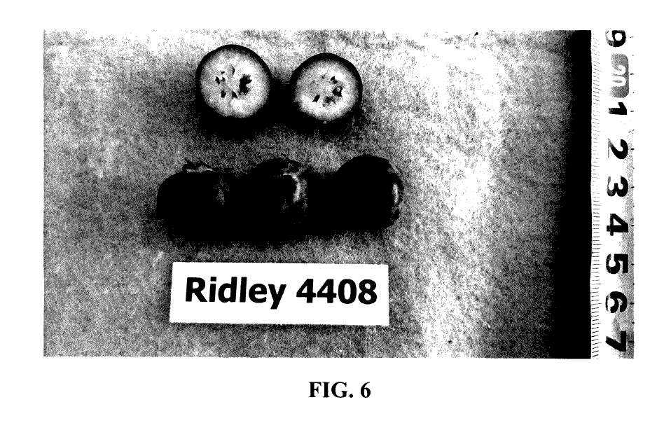

FIG. 7 is a photograph of the packaged fruit of the new variety ‘Ridley 4408’.

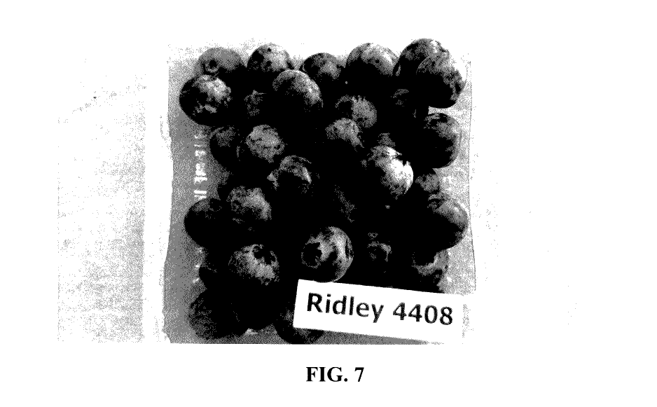

The colors in the photographs are as close as possible with the photographic and printing technology utilized. The color values cited in the detailed botanical description accurately describe the colors of the new blueberry.

### DETAILED BOTANICAL DESCRIPTION

The following detailed description sets forth the distinctive characteristics of ‘Ridley 4408’. The data that define these characteristics were collected from asexual reproductions of the original selection. Dimensions, sizes, colors, and other characteristics are approximations and averages set forth as accurately as possible. The plant history was taken on plants approximately 4 years of age, and the descriptions relate to plants grown in the field in Tabulam, New South Wales, Australia. Descriptions of fruit characteristics were made on fruit grown in Tabulam, New South Wales, Australia. Color designations are from Tabulam, New South Wales, Australia. Color notations are based on The Royal Horticultural Society Color Chart, of The Royal Horticultural Society, London, 2007 edition.
Classification:
   a. Family.—Ericaceae.
   b. Genus.—Vaccinium.
   c. Species.—hybrid.
   d. Common name.—Southern Highbush Blueberry.
Parentage:
   a. Female parent.—‘S01-15-01’ (unpatented).
   b. Male parent.—‘C00-09’ (U.S Plant Pat. No. 22,778).
   c. Market class.—A fruiting plant intended for use as fresh fruit for shipping, customer pick and processing markets and as a home garden plant.
Plant:
General:
   a. Parentage.—Female Parent — ‘S01-15-01.’ (unpatented). Male Parent — ‘C00-09’ (U.S. Plant Pat. No. 22,778).
   b. Plant height.—1.5 m.
   c. Plant width.—1.2 m.
   d. Growth habit.—Upright to semi-upright.
   e. Growth.—Medium vigor.
   f. Productivity.—Greater than 5 kg per season from 3-4 year old plants planted at 3.0 m×0.9 m density.
   g. Cold hardiness.—Has not been grown in all environments including harsh winter environments.
   h. Cold tolerance.—Cold tolerance is expected to be low.
   i. Chilling requirement.—Has not been grown in all environments and is typically grown as an evergreen crop where chilling hours are not important. ‘Ridley 4408’ is classed as ‘low chill’, typical of Southern Highbush Blueberry varieties with an estimated chilling requirement of 200 hours (not tested).
   j. Tolerance to disease.—Moderate resistance to root disease (phytophthora spp) and good resistance to blueberry rust.
   k. Leafing.—Vegetative bud burst is early to medium when grown as an evergreen in Australia.
   l. Twigginess.—Low.
Stem:
General:
   a. Suckering tendency.—Low. Plants typically have 5-7 major canes per plant from a base 25 cm in diameter on 4 year old plants.
   b. Mature cane color.—Color greyed orange 198C.
   c. Mature cane length.—0.8-0.9 m.
   d. Mature cane width.—1.2 cm.
   e. Bark texture.—Medium roughnees (typical of species).
   f. Fall color on new shoots.—Olive green (approximately yellow green RHS 146A-B) Colors are variable subject to conditions of environment and culture.
   g. Surface texture of new wood.—Smooth.
   h. Internode length on strong, new shoots.—15-20 mm.
   i. Fruiting wood.—To 15 cm in length.
Foliage:
General:
   a. Time of beginning of leaf bud burst.—Early to medium.
   b. Leaf color (top side).—Yellow green 147A.
   c. Leaf color (under side).—Yellow green 147C.
   d. Leaf arrangement.—Alternate.
   e. Leaf shape.—Elliptic.
   f. Leaf margins.—Entire.
   g. Undulation of margin.—Weak.
   h. Leaf venation.—Reticulate.
   i. Leaf apices.—Acuminate.
   j. Leaf bases.—Obtuse.
   k. Leaf length.—55-72 mm.
   l. Leaf width.—27-41 mm.
   m. Leaf length/width ratio.—Medium.
   n. Leaf nectaries.—Absent.
   o. Pubescence of upper side.—Absent to very weak.
   p. Pubescence of lower side.—Absent.
   q. Cross sectional profile.—Flat to very weakly concave.
   r. Longitudinal profile.—Straight.
   s. Attitude.—Semi-upright to horizontal.
Petioles:
   a. Length.—1.0 to 3.0 mm.
   b. Width.—2.0 mm.
   c. Color.—Yellow green 146C-147C.
Flowers:
General:
   a. Time of beginning of flowering.—Early to medium (mid May to early July at Lindendale, IN S W Australia).
   b. Time of 50% anthesis.—Mid June at Lindendale, NSW Australia.
   c. Flower shape.—Urceolate.
   d. Flower bud density.—Strong density.
   e. Flower fragrance.—Weak.
Corolla:
   a. Color.—White NN155D.
   b. Length.—10-12 mm.
   c. Width.—7-8 HMI.
   d. Aperture width.—2-3 mm.
   e. Anthocyanin coloration of corolla.—Absent.
   f. Corolla ridges.—Present.
   g. Protrusion of stigma.—Frequently present.
Inflorescence:
   a. Length.—35 to 45 mm.
   b. Diameter.—25 to 28 mm.
   c. Length of peduncle.—25-30 mm.
   d. Surface texture of peduncle.—Smooth.
   e. Color of peduncle.—146C.
   f. Length of pedicel.—8-12 mm.
   g. Surface texture of pedicel.—Smooth.
   h. Color of pedicel.—147C.
   i. Number of flowers per cluster.—10-14.
   j. Flower cluster density.—Strong.
Calyx (with sepals):
   a. Diameter.—12 mm.
   b. Color (sepals).—Green 138C to yellow green 147C.
Stamen:
   a. Length.—4-5 mm.
   b. Number per flower.—Approximately 10.
   c. Filament color.—Yellow green 144D.
Pistil:
   a. Length.—104 mm.
   b. Style: length.—5-6 mm
   c. Ovary color (exterior).—Yellow green 144D-147C.
Anther:
   a. Length.—3.0-3.5 mm.
   b. Number.—Approximately 10.
   c. Color.—Grayed orange 167C-D.
Pollen:
   a. Abundance.—Medium.
   b. Color.—Grayed orange 167C-D.
   c. Self-compatibility—90% fruit set (considered self-compatible)
Fruit:
General:
   a. Time of fruit ripening.—Early to medium (mid August to mid October at Lindendale, NSW Australia).
   b. Time of 50% maturity.—Mid-late September.
   c. Fruit development period.—55 days.
   d. Cluster density.—Medium to dense, 7-9 berries per cluster.
   e. Unripe fruit color.—Green 138A.
   f. Ripe Berry color.—Black 202A.
   g. Berry surface wax abundance.—Strong.
   h. Berry flesh color.—Grayed green 194A-195A.
   i. Berry weight.—2.7-3.0 g.
   j. Berry height from calyx to scar.—13-15 mm.
   k. Berry diameter.—18 mm.
   l. Berry shape.—Round with occasional slight oblate.
   m. Fruit stem scar.—Small (dry).
   n. Sweetness when ripe.—High.
   o. Firmness when ripe.—Firm.
   p. Acidity when ripe.—High.
   q. Storage quality.—Good, lasted 10 weeks at 2 degrees Celsius in tests. Not tested with modified atmosphere storage yet.
   r. Suitability for mechanical harvesting.—Firm fruit firmness, good ease of detachment by shaking, strong blush and fruit shape suited to mechanical harvesting.
   s. Self-fruitfulness.—90% fruit set (considered self-compatible).
   t. Uses.—Fresh fruit for shipping, customer pick and processing markets and as a home garden plant.
Seed:
General:
   a. Seed abundance in fruit.—Abundant.
   b. Seed color.—Greyed orange 174B.
   c. Seed dry weight.—About 65 seeds/1 mg.
   d. Seed length.—1.6-1.8 mm.

### COMPARISON BETWEEN PARENTAL AND COMMERCIAL CULTIVARS

| Organ | Context | Ridley 4408 | Ridley 4609 | Ridley 1812 | Ridley 1403 | C00-09 |
|---|---|---|---|---|---|---|
| Plant | vigour | medium | medium | medium | strong | strong |
| Plant | growth habit | Upright to | Upright to | upright | upright to | upright to |
| | | semi upright | semi upright | | semi-upright | semi-upright |
| One-year- | colour | green | green | green | green | green |
| old shoot | | | | | | |
| One-year- | length of | medium | medium | medium | medium | medium |
| old shoot | internode | | | | | |
| Leaf | length | long | medium to | long to very | long to very | long |
| | | | long | long | long | |
| Leaf | width | broad | broad | broad | broad | broad |
| Leaf | shape | elliptic | elliptic | elliptic | elliptic | elliptic |
| Leaf | colour of | green | green | green | green | green |
| | upper side | | | | | |
| Leaf | intensity of | medium | medium | medium | medium | dark |
| | green colour | | | | | |
| | on upper | | | | | |
| | side | | | | | |
| Leaf | margin | entire | entire | entire | entire | entire |
| Flower bud | anthocyanin | weak | weak | weak | weak | weak |
| | coloration | | | | | |
| Flower | shape of | urceolate | urceolate | urceolate | urceolate | urceolate |
| | corolla | | | | | |
| Flower | size of | medium | medium | medium | medium to | medium to |
| | corolla tube | | | | large | large |
| Flower | anthocyanin | absent or | absent or | absent or | absent or | absent or |
| | colouration | very weak | very weak | very weak | very weak | very weak |
| | of corolla | | | | | |
| | tube | | | | | |
| Flower | ridges on | present | present | present | present | present |
| | corolla tube | | | | | |
| Fruit cluster | density | medium to | | | medium to | |
| | | dense | medium | medium | dense | medium |
| Unripe fruit | intensity of | light | light | light | light | light |
| | green colour | | | | | |
| Fruit | size | medium | medium | very large | very large | very large |
| Fruit | shape in | round | oblate | oblate | round | oblate |
| | longitudinal | | | | | |
| | section | | | | | |
| Fruit | attitude of | erect | erect | erect | erect | erect |
| | sepals | | | | | |
| Fruit | diameter of | large | large | large to very | large | large |
| | calyx basin | | | large | | |
| Fruit | depth of | deep | deep | deep to very | deep | deep |
| | calyx basin | | | deep | | |
| Fruit | intensity of | strong | weak to | weak to | medium | medium to |
| | bloom | | medium | medium | | strong |
| Fruit | colour of | dark blue | dark blue | dark blue | dark blue | dark blue |
| | skin | | | | | |
| Fruit | firmness | firm | firm | medium | medium | very firm |
| Fruit | sweetness | high | medium to | medium to | low to | high |
| | | | high | high | medium | |
| Fruit | acidity | high | medium to | medium to | medium to | low |
| | | | high | high | high | |
| Time of | vegetative | early to | late | medium | early to | late |
| | bud burst | medium | | | medium | |
| Time of | beginning of | early to | late | late | very early | late |
| | flowering | medium | | | | |
| Time of | beginning of | early to | late | late | early to | late |
| | fruit | medium | | | medium | |
| | ripening | | | | | |

1. A new and distinct variety of blueberry plant named ‘RIDLEY 4408’, substantially as illustrated and described herein.
